(19) United States
(12) Patent Application Publication
(10) Pub. No.: US 2026/0174968 A1
(43) Pub. Date: Jun. 25, 2026

(54) POGO PIN AND MANUFACTURING METHOD THEREFOR

(71) Applicant: OKINS ELECTRONICS CO.,LTD, Uiwang-si, Gyeonggi-do (KR)

(72) Inventors: Sang Hoon CHA, Hwaseong-si (KR); Jin Kook JUN, Gunpo-si (KR); Eun Hyeong PYO, Bucheon-si (KR); Soo Ho LEE, Seoul (KR)

(21) Appl. No.: 19/128,900

(22) PCT Filed: Nov. 10, 2022
(86) PCT No.: PCT/KR2022/017637

§ 371 Date: May 9, 2025

(51) Int. Cl. A61M 5/315; G01R 1/04; G01R 1/067; G01R 1/073; G01R 1/18; H01R 13/24; H01R 13/631

(52) U.S. Cl. CPC A61M 5/31515; G01R 1/0466; G01R 1/06722; G01R 1/06738; G01R 1/07378; G01R 1/18; H01R 13/2421; H01R 13/631

(57) ABSTRACT

Disclosed are a pogo pin that enables a pre-designed impedance value to be more easily matched, and a manufacturing method therefor. The pogo pin comprises: a plunger part; a barrel into which at least a portion of the plunger part is inserted; a housing in which the barrel is accommodated; and an insulating ring made of a dielectric material and located between the outer peripheral surface of a portion of the plunger part exposed to the outside of the barrel and the inner peripheral surface of the housing.

### BACKGROUND

### Technical Field

The present disclosure relates to a pogo pin and a manufacturing method therefor, and more particularly, to a pogo pin and a manufacturing method therefor that enable a pre-designed impedance value to be more easily matched.

### Description of the Related Art

A pogo pin refers to a device that is provided between a semiconductor device and a test apparatus to electrically connect the semiconductor device to the test apparatus. In order to test the electrical characteristics of a semiconductor device, it is required that the semiconductor device and the test apparatus be electrically connected in a stable manner, and in this case, the pogo pin plays a key role.

The pogo pin includes a plunger part that comes into contact with a semiconductor device or a test apparatus, a barrel into which a portion of the plunger part is inserted, and a housing that accommodates the barrel therein. The inner peripheral surface of the housing and the outer peripheral surface of the barrel are spaced apart from each other for insulation. In this case, the housing may be coupled to the barrel in a state spaced apart from the outer peripheral surface of the barrel by means of an insulating ring.

The insulating ring is located between the inner peripheral surface of the housing and the outer peripheral surface of the plunger part. However, since the dielectric constant of a typical insulating ring is generally greater than that of air, its outer diameter needs to be increased in order to maintain a constant impedance value. Accordingly, it is not possible to maintain a constant impedance value in each section without increasing the outer diameter of the housing or decreasing the outer diameter of the barrel. In other words, it is difficult to achieve proper impedance value matching.

In addition, an increase in the outer diameter of the insulating ring may also cause a change in the outer diameter of the housing. This complicates the manufacturing and assembly processes of the components of the pogo pin.

Korean Patent Registration No. 10-2092674 discloses a test probe assembly and a test socket. More specifically, it discloses a test probe assembly and a test socket in which a probe support member is inserted between a pipe insertion part and an extension part.

However, in this type of test probe assembly and test socket, the outer diameters of the probe support member and the extension part are not maintained constant, and an increase in the outer diameter of the probe support member is required to achieve proper impedance value matching.

(patent Document 1) Korean Patent Registration No. 10-2092674 (2020.3.24.)

### BRIEF SUMMARY

### Technical Problem

The present disclosure is directed to providing a pogo pin and a method for manufacturing the same, which enable more easily matching a predetermined impedance value.

The present disclosure is also directed to providing a pogo pin and a method for manufacturing the same, in which an insulating ring is formed to extend with a constant diameter.

The present disclosure is also directed to providing a pogo pin and a method for manufacturing the same, in which an assembly process can be further simplified.

### Technical Solution

In order to achieve the above objects, the pogo pin according to an embodiment of the present disclosure includes a plunger part configured to come into contact with an external terminal; a barrel into which at least a portion of the plunger part is inserted; a housing in which the barrel is received; and an insulating ring positioned between an outer peripheral surface of a portion of the plunger part exposed to the outside of the barrel and an inner peripheral surface of the housing, the insulating ring being formed of a dielectric material.

In addition, the insulating ring may have an outer diameter that is equal to or smaller than an inner diameter of the housing.

In addition, the insulating ring may have an inner diameter that is equal to or greater than an outer diameter of the portion of the plunger part.

In addition, the plunger part may include a large-diameter portion received inside the barrel; and a small-diameter portion formed to have a diameter smaller than that of the large-diameter portion and exposed to the outside of the barrel.

In addition, the insulating ring may be disposed to surround an outer peripheral surface of the small-diameter portion.

In addition, the housing may have locking steps formed by both ends of the housing being radially bent inward.

In addition, the locking steps may be formed by a caulking process.

In addition, a portion of each locking step that contacts the insulating ring may be formed in a shape corresponding to an outer peripheral surface of the insulating ring.

In addition, the insulating ring may be provided at each of both ends of the housing.

In addition, air may be filled between the two insulating rings provided at both ends of the housing.

In addition, the present disclosure provides a method for manufacturing a pogo pin, including (a) coupling an elastic member and a plunger part in an internal space of a barrel; (b) fitting a portion of the plunger part exposed to the outside of the barrel through an insulating ring; and (c) inserting the barrel and the insulating ring into an internal space of a housing.

In addition, after the step (c), (d) caulking both ends of the housing may be performed.

### Advantageous Effect

Among the various effects of the present disclosure, the effects that can be obtained through the above-described solution are as follows.

First, the pogo pin includes a plunger part, a barrel into which a portion of the plunger part is inserted, a housing in which the barrel is received, and an insulating ring positioned between the plunger part and the housing. In this case, the insulating ring is disposed to surround an outer peripheral surface of a portion of the plunger part exposed outside the barrel. That is, the insulating ring is fitted through a driving section of the plunger part.

Therefore, an impedance value can be adjusted by controlling the inner diameter of the insulating ring and the outer diameter of the portion of the plunger part. Accordingly, while the housing is formed to extend with a constant outer diameter, the impedance can also be maintained at a constant value over the entire section. In summary, matching a pre-designed impedance value becomes easier. Furthermore, the RF characteristics of the pogo pin can be further improved.

In addition, locking steps are provided at both ends of the housing, the locking steps being formed by bending radially inward. In this case, the locking steps are formed through a caulking process.

Therefore, the insulating ring may be formed to extend with a constant outer diameter. Accordingly, an impedance value over the entire section of the insulating ring can be maintained constant, and a change in the impedance value due to a variation in the outer diameter can be excluded from consideration during design. As a result, the manufacturing process of the pogo pin can be further simplified.

In addition, as described above, the housing and the insulating ring are formed to extend while maintaining their respective outer diameters constant. That is, the shapes of the housing the insulating ring can be further simplified.

Therefore, the manufacturing processes of the housing and the insulating ring can be further simplified. Furthermore, the assembly process of the plunger part, the barrel, the housing, and the insulating ring can be further simplified.

### BRIEF DESCRIPTION OF THE SEVERAL VIEWS OF THE DRAWINGS

FIG. 1 is a cross-sectional view illustrating a pogo pin according to an embodiment of the present disclosure.

FIG. 2 is an exploded cross-sectional view illustrating the components of the pogo pin shown in FIG. 1.

FIG. 3 is a cross-sectional view illustrating the states before and after movement of the plunger part of the pogo pin shown in FIG. 1.

FIG. 4 is an enlarged cross-sectional view illustrating the plunger part and the insulating ring provided in the pogo pin shown in FIG. 1.

FIG. 5 is a flowchart illustrating a method for manufacturing a pogo pin according to an embodiment of the present disclosure.

### DETAILED DESCRIPTION

Hereinafter, a pogo pin 1 and a manufacturing method therefor according to an embodiment of the present disclosure will be described in more detail with reference to the drawings.

In the following description, in order to clarify the features of the present disclosure, descriptions of some components may be omitted.

In this specification, even in different embodiments, the same reference numerals will designate the same elements, and a redundant description thereof will be omitted.

The accompanying drawings are only for easy understanding of the embodiments disclosed herein, and the technical ideas disclosed herein are not limited by the accompanying drawings.

Expressions in the singular include plural expressions unless the context clearly indicates otherwise.

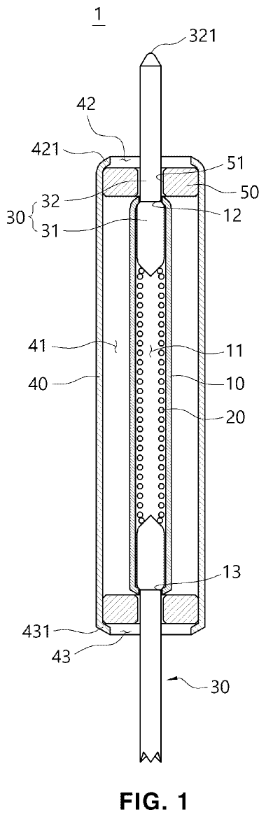

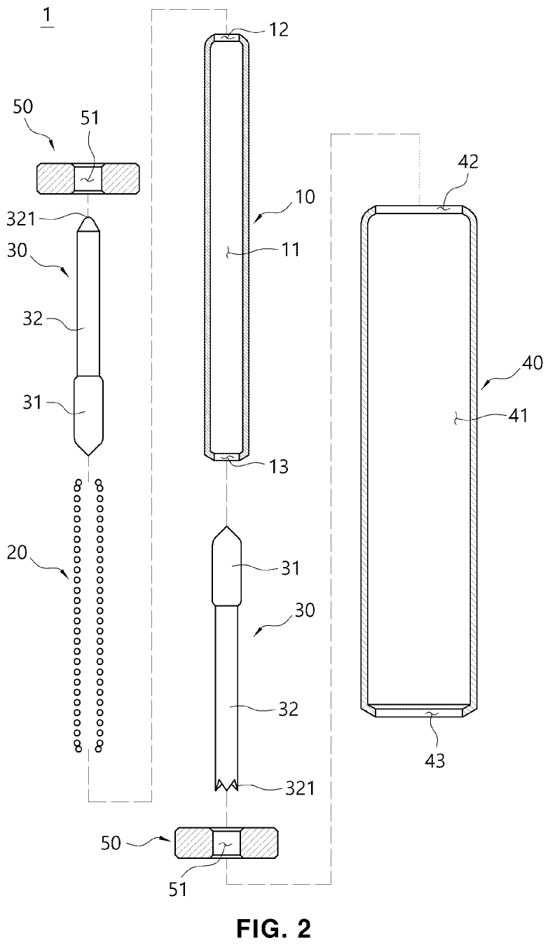

Hereinafter, a pogo pin 1 according to an embodiment of the present disclosure will be described with reference to FIGS. 1 and 2.

The pogo pin 1 is provided between a semiconductor device and a test apparatus to electrically connect the semiconductor device to the test apparatus. To this end, the pogo pin 1 is arranged to be in contact with the semiconductor device and the test apparatus, respectively.

The pogo pin 1 extends in a direction from the semiconductor device toward the test apparatus or in a direction from the test apparatus toward the semiconductor device.

In the illustrated embodiment, the pogo pin 1 includes a barrel 10, an elastic member 20, a plunger part 30, a housing 40, and an insulating ring 50.

The barrel 10 is configured such that a portion of the plunger part 30, which will be described later, is inserted therein and guides the movement of the plunger part 30.

The barrel 10 is formed in a columnar shape extending in one direction. In an exemplary embodiment, both ends of the barrel 10 may be bent inward in the radial direction. This is to prevent unintended separation of the elastic member 20 and the plunger part 30.

A plunger receiving portion 11 is formed inside the barrel 10. The elastic member 20 and a portion of the plunger part 30, which will be described later, are inserted into the plunger receiving portion 11.

An upper plunger hole 12 and a lower plunger hole 13 are formed at the top and bottom of the barrel 10, respectively. A detailed description thereof will be provided later in conjunction with the description of the plunger part 30.

The elastic member 20 is received in the plunger receiving portion 11 of the barrel 10.

The elastic member 20 provides a restoring force when the plunger part 30, which will be described later, moves.

The elastic member 20 is positioned at the center of the plunger receiving portion 11.

The elastic member 20 extends in the same direction as the extending direction of the barrel 10. In addition, the elastic member 20 applies a restoring force along the extending direction.

In the illustrated embodiment, the elastic member 20 is formed as a spring. However, the elastic member 20 is not limited to the illustrated form and may be formed in various shapes capable of providing a restoring force to the plunger part 30.

The plunger part 30 is in contact with both ends of the elastic member 20.

The plunger part 30 is the portion of the pogo pin 1 that comes into direct contact with the semiconductor device or the test apparatus.

The plunger part 30 extends in the same direction as the extending direction of the barrel 10. In addition, a portion of the plunger part 30 is inserted into the plunger receiving portion 11 of the barrel 10, and the remaining portion is exposed to the outside of the barrel 10.

Accordingly, the plunger part 30 may reciprocate along the extending direction of the barrel 10.

The plunger part 30 is arranged to be in contact with one end of the elastic member 20. Accordingly, the plunger part 30 receives a restoring force from the elastic member 20 during movement. A detailed description thereof will be described later.

The plunger part 30 is formed of an electrically conductive material. In an exemplary embodiment, the plunger part 30 is formed of a metallic material.

A plurality of plunger parts 30 may be provided. In the illustrated embodiment, two plunger parts 30 are provided. Each of the plunger parts 30 is inserted through and coupled to the upper plunger hole 12 or the lower plunger hole 13.

In the illustrated embodiment, the plunger part 30 includes a large-diameter portion 31 and a small-diameter portion 32.

The large-diameter portion 31 is a part of the plunger part 30 that comes into direct contact with the elastic member 20.

The large-diameter portion 31 is inserted into the plunger receiving portion 11 of the barrel 10. Accordingly, it is preferable that the outer diameter of the large-diameter portion 31 be equal to or smaller than the inner diameter of the barrel 10.

The large-diameter portion 31 may reciprocate along the extending direction of the barrel 10 within the plunger receiving portion 11. In the illustrated embodiment, the large-diameter portion 31 is prevented from being released to the outside of the barrel 10 by bent portions formed at both ends of the barrel 10.

The small-diameter portion 32 is a part of the plunger part 30 that is exposed to the outside of the barrel 10. In the illustrated embodiment, the small-diameter portion 32 is inserted through and coupled to the upper plunger hole 12 or the lower plunger hole 13.

The small-diameter portion 32 is formed to extend from one end of the large-diameter portion 31 opposite to the elastic member 20 in a direction away from the elastic member 20.

The small-diameter portion 32 is formed with a diameter smaller than that of the large-diameter portion 31. It is preferable that the outer diameter of the small-diameter portion 32 be equal to or smaller than the inner diameter of the end portion of the barrel 10.

A contact tip portion 321, which is arranged to be in contact with the semiconductor device or the test apparatus, is formed at one end of the small-diameter portion 32 opposite to the large-diameter portion 31.

The barrel 10, which is coupled with the elastic member 20 and the plunger part 30, is received in an inner space of the housing 40.

The housing 40 forms the exterior of the pogo pin 1 and electrically insulates the plunger part 30 from the surrounding environment, excluding the semiconductor device or the test apparatus.

The housing 40 is positioned on the radial outside of the barrel 10. Accordingly, it will be understood that the inner diameter of the housing 40 is formed to be larger than the outer diameter of the barrel 10.

In addition, the inner peripheral surface of the housing 40 is spaced apart from the outer peripheral surface of the barrel 10. In an exemplary embodiment, air is filled between the inner peripheral surface of the housing 40 and the outer peripheral surface of the barrel 10.

The housing 40 is formed in a columnar shape extending in one direction. In this case, the housing 40 extends in the same direction as the extending direction of the barrel 10.

The housing 40 is formed to have a length in the longitudinal direction that is greater than the length of the barrel 10. In addition, the upper end of the housing 40 is positioned above the upper end of the barrel 10, and the lower end is positioned below the lower end of the barrel 10. In other words, the barrel 10 is inserted into and enclosed within the housing 40.

A barrel receiving portion 41 is formed inside the housing 40. The barrel 10, the elastic member 20, and the plunger part 30 are inserted into the barrel receiving portion 41.

An upper opening 42 and a lower opening 43 are formed at the upper end and the lower end of the housing 40, respectively.

An upper locking step 421 and a lower locking step 431 are formed at the respective boundaries of the upper opening 42 and the lower opening 43.

The upper locking step 421 and the lower locking step 431 are formed by bending the upper end or the lower end of the housing 40 radially inward. In an exemplary embodiment, the upper locking step 421 and the lower locking step 431 are formed through a caulking process.

The insulating ring 50 is positioned between the housing 40 and the plunger part 30.

The insulating ring 50 fixes the plunger part 30 to be positioned at the center of the housing 40.

The insulating ring 50 is positioned between the outer peripheral surface of the small-diameter portion 32 of the plunger part 30 and the inner peripheral surface of the housing 40. In addition, the insulating ring 50 is disposed to surround the outer peripheral surface of the small-diameter portion 32. That is, the insulating ring 50 is fitted through the driving section of the plunger part 30. A detailed description thereof will be described later.

The insulating ring 50 is located on an upper side or a lower side of the barrel 10.

The, the inner diameter of the insulating ring 50 is not affected by the outer diameter of the barrel 10.

The insulating ring 50 is formed in a cylindrical shape in which a hollow portion 51 is formed at its central portion. The extending direction of the insulating ring 50 is formed to be parallel to the extending directions of the plunger part 30 and the housing 40.

The outer diameter of the insulating ring 50 is formed to be equal to or smaller than the inner diameter of the housing 40. In addition, the inner diameter of the insulating ring 50 is formed to be equal to or greater than the outer diameter of the small-diameter portion 32 of the plunger part 30.

In an exemplary embodiment, the outer peripheral surface of the insulating ring 50 may come into contact with a portion of the upper locking step 421 and the lower locking step 431, which are formed through a caulking process, and may be formed in a shape corresponding to the upper locking step 421 and the lower locking step 431.

Therefore, the insulating ring 50 may be formed to extend with a constant outer diameter. Accordingly, an impedance value over the entire section of the insulating ring 50 may be maintained constant, and a change in the impedance value due to a variation in the outer diameter may be excluded from consideration during design. As a result, the manufacturing process of the pogo pin 1 may be further simplified.

In another embodiment, the insulating ring 50 may be inserted into the housing 40 through a press-fitting process. In the above embodiment, locking steps 421 and 431 may not be formed at both ends of the housing 40.

A plurality of insulating rings 50 may be provided. In the illustrated embodiment, two insulating rings 50 are provided in total, one at the upper end and the other at the lower end of the housing 40. In an exemplary embodiment, air may be filled between the two insulating rings 50 provided at both ends of the housing 40.

The insulating ring 50 is formed of an electrically insulating material. In an exemplary embodiment, the insulating ring 50 is formed of a PEEK (polyether ether ketone) material. In another embodiment, the insulating ring 50 is formed of a dielectric material having a higher dielectric constant than air.

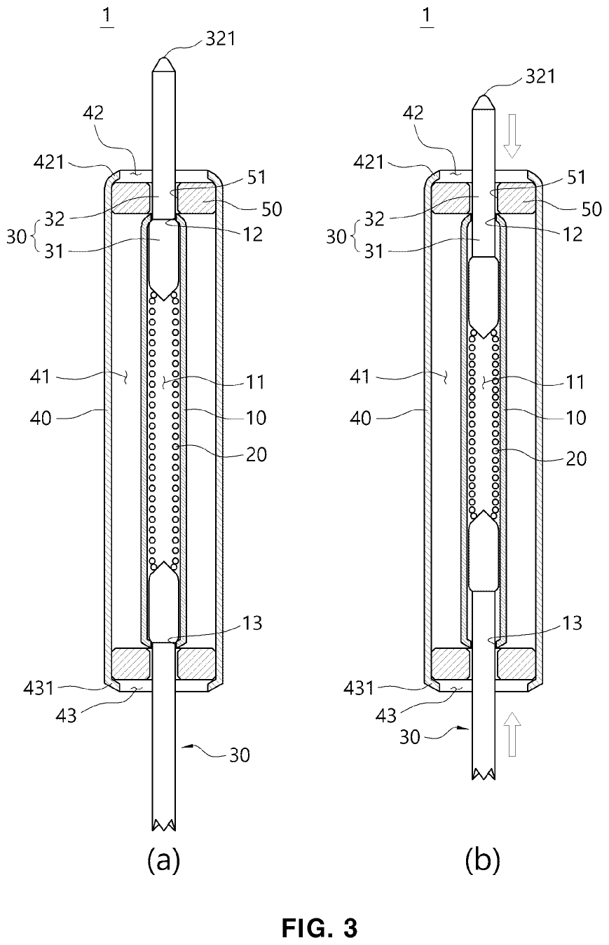

Hereinafter, a movement process of the plunger part 30 will be described with reference to FIG. 3.

The pogo pin 1 is provided between a semiconductor device and a test apparatus, and its length may be adjusted in response to a distance between the semiconductor device and the test apparatus. The length adjustment process may be achieved by the movement of the plunger part 30.

In the illustrated embodiment, two plunger parts 30 are arranged to face each other with the elastic member 20 interposed therebetween. A contact tip portion 321, which comes into contact with the semiconductor device or the test apparatus, is formed at one end of each plunger part 30 opposite to the elastic member 20.

FIG. 3(a) illustrates the pogo pin 1 in a state before the movement of the plunger part 30, in which the elastic member 20 is at its maximum length.

FIG. 3(b) illustrates the pogo pin 1 in a state after the movement of the plunger part 30. More specifically, FIG. 3(b) illustrates the pogo pin 1 in a state in which the plunger part 30 has moved toward the elastic member 20.

When the plunger part 30 moves toward the elastic member 20, the elastic member 20 is compressed and applies a restoring force to the plunger part 30. As a result, the plunger part 30 may be brought into close contact with the semiconductor device or the test apparatus.

In the above process, it can be seen that the small-diameter portion 32 of the plunger part 30 is exposed to the upper side or the lower side of the barrel 10. That is, a driving section of the plunger part 30 is formed in a space on the upper side or the lower side of the barrel 10.

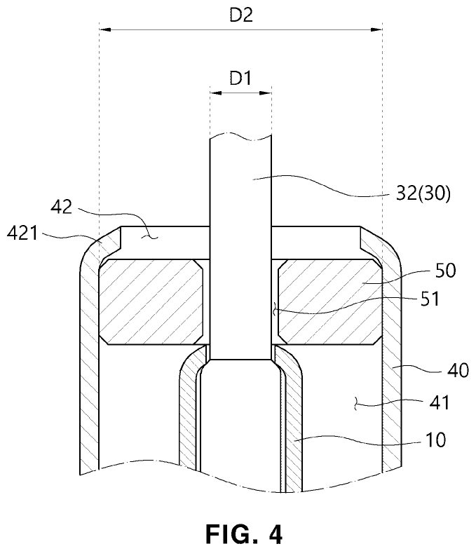

Hereinafter, the relationship among the insulating ring 50, the plunger part 30, and the housing 40 will be described with reference to FIG. 4.

Hereinafter, a diameter of the small-diameter portion 32 of the plunger part 30 is defined as a first diameter D1, and an outer diameter of the insulating ring 50 is defined as a second diameter D2.

It is preferable that the first diameter D1 be equal to or smaller than the inner diameter of the insulating ring 50. It is also preferable that the second diameter D2 be equal to or smaller than the inner diameter of the housing 40.

The insulating ring 50 is fitted through a driving section of the plunger part 30.

Specifically, the insulating ring 50 is inserted between the outer peripheral surface of the small-diameter portion 32 of the plunger part 30 and the inner peripheral surface of the housing 40.

Therefore, the impedance value may be adjusted by controlling the first diameter D1 and the second diameter D2. Accordingly, while the housing 40 is formed to extend with a constant outer diameter, the impedance may also be maintained at a constant value over the entire section. In summary, matching a pre-designed impedance value becomes easier. Furthermore, the RF characteristics of the pogo pin 1 may be further improved.

In contrast, when the insulating ring 50 is coupled through a non-driving section of the plunger part 30, that is, an outer peripheral surface of the barrel 10, it is difficult to maintain a constant impedance value for each section without increasing the outer diameter of the housing 40 or decreasing the outer diameter of the barrel 10.

In addition, the housing 40 and the insulating ring 50 are each formed to extend while maintaining a constant outer diameter. That is, the shapes of the housing 40 and the insulating ring 50 may be further simplified.

Therefore, the manufacturing processes of the housing 40 and the insulating ring 50 may be further simplified. Furthermore, the assembly process of the plunger part 30, the barrel 10, the housing 40, and the insulating ring 50 may be further simplified.

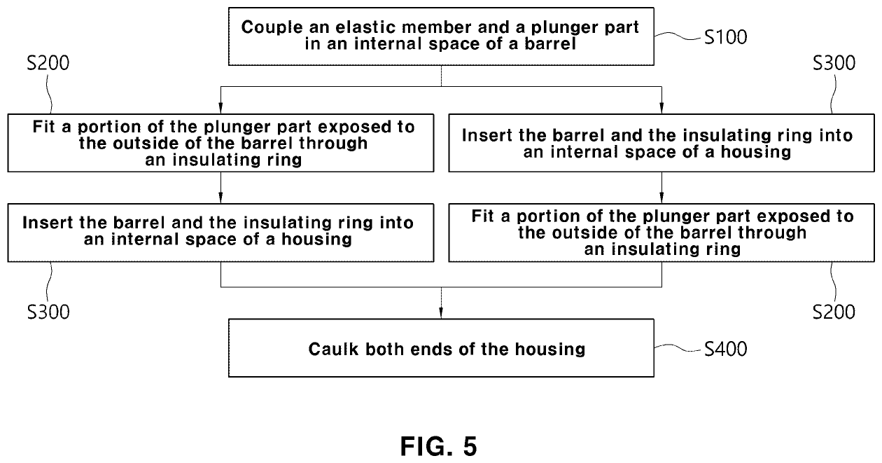

Hereinafter, a manufacturing method of the pogo pin 1 according to an embodiment of the present disclosure will be described with reference to FIG. 5.

The manufacturing method of the pogo pin 1 according to an embodiment of the present disclosure includes: a step (S100) of coupling the elastic member 20 and the plunger part 30 in an inner space of the barrel 10; a step (S200) of fitting a portion of the plunger part 30 exposed to the outside of the barrel 10 through the insulating ring 50; a step (S300) of inserting the barrel 10 and the insulating ring 50 into an inner space of the housing 40; and a step (S400) of caulking both ends of the housing 40.

First, the step (S100) of coupling the elastic member 20 and the plunger part 30 in the inner space of the barrel 10 will be described.

The barrel 10 is formed in a cylindrical shape extending with a constant diameter, and the plunger receiving portion 11 is formed therein. The elastic member 20 is received in the central portion of the plunger receiving portion 11. In addition, large-diameter portions 31 of the plunger part 30 are inserted into both ends of the plunger receiving portion 11.

In this case, the large-diameter portions 31 are arranged to be in contact with the elastic member 20. In addition, the small-diameter portion 32 of the plunger part 30 is arranged to be exposed to the outside of the barrel 10.

When the elastic member 20 and the plunger part 30 are coupled inside the barrel 10, a step (S200) of fitting a portion of the plunger part 30 exposed to the outside of the barrel 10 through the insulating ring 50 and a step (S300) of inserting the barrel 10 and the insulating ring 50 into an inner space of the housing 40 are performed.

First, the step (S200) of fitting a portion of the plunger part 30 exposed to the outside of the barrel 10 through the insulating ring 50 will be described.

A cylindrical insulating ring 50 having a hollow portion 51 formed therein is formed. Subsequently, the small-diameter portion 32 of the plunger part 30 exposed to the outside of the barrel 10 is fitted through the hollow portion 51 of the insulating ring 50. In this case, it is preferable that the outer diameter of the small-diameter portion 32 be equal to or smaller than the inner diameter of the hollow portion 51.

Next, the step (S300) of inserting the barrel 10 and the insulating ring 50 into an inner space of the housing 40 will be described.

When the plunger part 30 and the insulating ring 50 are coupled, a structure including the barrel 10 and the peak ring is inserted into an inner space of the housing 40. At this time, the outer peripheral surface of the barrel 10 is spaced apart from the inner peripheral surface of the housing 40, and the barrel 10 is received and enclosed within the housing 40.

In addition, the insulating ring 50 is formed to have an outer diameter equal to or smaller than the inner diameter of the housing 40.

However, the order between the step (S200) of fitting a portion of the plunger part 30 exposed to the outside of the barrel 10 through the insulating ring 50 and the step (S300) of inserting the barrel 10 and the insulating ring 50 into an inner space of the housing 40 is variable, and either one of the steps may be performed first, followed by the other.

Finally, a step (S400) of caulking both ends of the housing 40 is performed.

Immediately after the insulating ring 50 is inserted into the upper and lower ends of the barrel receiving portion 41 within the housing 40, a predetermined height difference exists between the end of the housing 40 and the end of the insulating ring 50. Subsequently, as both ends of the housing 40 are caulked, the ends are bent radially inward and locking steps 421 and 431 are formed.

Accordingly, the upper end or the lower end of the insulating ring 50 is supported by the locking steps 421 and 431 of the housing 40, and unintended separation of the insulating ring 50 to the outside of the housing 40 can be prevented.

Although the present disclosure has been described above with reference to preferred exemplary embodiments thereof, the present disclosure is not limited to the configurations of the above-described embodiments.

In addition, the present disclosure may be variously modified and changed without departing from the idea and scope of the present disclosure described in the following claims by those skilled in the art to which the present disclosure pertains.

Furthermore, the embodiments may be configured by selectively combining all or some of the embodiments so that various modifications may be made thereto.

### DESCRIPTION OF SYMBOLS

1: pogo pin
10: barrel
11: plunger receiving portion
12: upper plunger hole
13: lower plunger hole
20: elastic member
30: plunger part
31: large-diameter portion
32: small-diameter portion
321: contact tip portion
40: housing
41: barrel receiving portion
42: upper opening
421: upper locking step
43: lower opening
431: lower locking step
50: insulating ring
51: hollow portion

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A pogo pin, comprising:
a plunger part configured to come into contact with an external terminal;
a barrel into which at least a portion of the plunger part is inserted;
a housing in which the barrel is received; and
an insulating ring positioned between an outer peripheral surface of a portion of the plunger part exposed to the outside of the barrel and an inner peripheral surface of the housing, the insulating ring being formed of a dielectric material.

2. The pogo pin of claim 1, wherein the insulating ring has an outer diameter that is equal to or smaller than an inner diameter of the housing.

3. The pogo pin of claim 2, wherein the insulating ring has an inner diameter that is equal to or greater than an outer diameter of the portion of the plunger part.

4. The pogo pin of claim 1,
wherein the plunger part comprises:
a large-diameter portion received inside the barrel; and
a small-diameter portion formed to have a diameter smaller than that of the large-diameter portion and exposed to the outside of the barrel.

5. The pogo pin of claim 4, wherein the insulating ring is disposed to surround an outer peripheral surface of the small-diameter portion.

6. The pogo pin of claim 1, wherein the housing has locking steps formed by both ends of the housing being radially bent inward.

7. The pogo pin of claim 6, wherein the locking steps are formed by a caulking process.

8. The pogo pin of claim 6, wherein a portion of each locking step that contacts the insulating ring is formed in a shape corresponding to an outer peripheral surface of the insulating ring.

9. The pogo pin of claim 1, wherein the insulating ring is provided at each of both ends of the housing.

10. The pogo pin of claim 9, wherein air is filled between the two insulating rings provided at both ends of the housing.

11. A method for manufacturing a pogo pin, comprising:
(a) coupling an elastic member and a plunger part in an internal space of a barrel;
(b) fitting a portion of the plunger part exposed to the outside of the barrel through an insulating ring; and
(c) inserting the barrel and the insulating ring into an internal space of a housing.

12. The method for manufacturing a pogo pin of claim 11, further comprising: after the step (c),
(d) caulking both ends of the housing.
